(19) United States
(12) Patent Application Publication
(10) Pub. No.: US 2025/0255625 A1
(43) Pub. Date: Aug. 14, 2025

(54) ASPIRATION CATHETER HAVING DISTAL SIDEWALL OPENING

(71) Applicant: Covidien LP, Mansfield, MA (US)

(72) Inventors: Daniel J. Deen, Long Beach, CA (US); John I. Goumas, Hacienda Heights, CA (US)

(21) Appl. No.: 19/053,129

(22) Filed: Feb. 13, 2025

Related U.S. Application Data
(60) Provisional application No. 63/552,816, filed on Feb. 13, 2024.

(51) Int. Cl. A61B 17/22; A61B 17/00

(52) U.S. Cl. CPC A61B 17/22; A61B 17/00234; A61B 2017/00238; A61B 2017/00544; A61B 2017/22079; A61B 2217/005

(57) ABSTRACT

Systems and methods for removal of clot material from vessel lumens are disclosed. A thrombectomy system can include a tubular member having a proximal end region configured to be disposed extracorporeally and a distal end region configured to be disposed at an intravascular treatment site at or adjacent to a thrombus. The tubular member has a sidewall defining a lumen extending therethrough from the proximal end region to the distal end region. The sidewall has an opening in the distal end region, the opening including a proximal portion spanning a first width and a distal portion spanning a second width greater than the first width. The thrombectomy system can additionally include a suction source configured to be fluidically coupled to the lumen.

### CROSS-REFERENCE TO RELATED APPLICATIONS

This application claims the benefit of and priority to U.S. Provisional Patent Application Ser. No. 63/552,816 filed Feb. 13, 2024, the entire disclosure of which is incorporated by reference herein.

### TECHNICAL FIELD

The present technology relates generally to systems and methods for removing obstructions from body lumens. Some embodiments of the present technology relate to aspiration catheters having distal sidewall openings.

### BACKGROUND

Many medical procedures use medical device(s) to remove an obstruction (such as clot material) from a body lumen, vessel, or other organ. An inherent risk in such procedures is that mobilizing or otherwise disturbing the obstruction can potentially create further harm if the obstruction or a fragment thereof dislodges from the retrieval device. If all or a portion of the obstruction breaks free from the device and flows downstream, it is highly likely that the free material will become trapped in smaller and more tortuous anatomy. In many cases, the physician will no longer be able to use the same retrieval device to again remove the obstruction because the device may be too large and/or immobile to move the device to the site of the new obstruction.

Procedures for treating ischemic stroke by restoring flow within the cerebral vasculature are subject to the above concerns. The brain relies on its arteries and veins to supply oxygenated blood from the heart and lungs and to remove carbon dioxide and cellular waste from brain tissue. Blockages that interfere with this blood supply eventually cause the brain tissue to stop functioning. If the disruption in blood occurs for a sufficient amount of time, the continued lack of nutrients and oxygen causes irreversible cell death. Accordingly, it is desirable to provide immediate medical treatment of an ischemic stroke.

To access the cerebral vasculature, a physician typically advances a catheter from a remote part of the body (typically a leg) through the abdominal vasculature and into the cerebral region of the vasculature. Once within the cerebral vasculature, the physician deploys a device for retrieval of the obstruction causing the blockage, for example an aspiration catheter. Concerns about dislodged obstructions or the migration of dislodged fragments increases the duration of the procedure at a time when restoration of blood flow is paramount. Furthermore, a physician might be unaware of one or more fragments that dislodge from the initial obstruction and cause blockage of smaller more distal vessels. Accordingly, there remains a need for improved devices and methods that can remove occlusions from body lumens and/or vessels.

### SUMMARY

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.

Clause 1. A thrombectomy system comprising: a thrombectomy device including a tubular member having a proximal end region configured to be disposed extracorporeally and a distal end region configured to be disposed at an intravascular treatment site at or adjacent to a thrombus, the tubular member comprising: a sidewall defining a lumen extending therethrough from the proximal end region to the distal end region; and an opening in the sidewall in the distal end region, the opening including a proximal portion spanning a first width and a distal portion spanning a second width greater than the first width; and a suction source configured to be fluidically coupled to the lumen.

Clause 2. The thrombectomy system of Clause 1, further comprising a cover at least partially overlying the proximal portion of the opening.

Clause 3. The thrombectomy system of Clause 2, wherein the cover comprises a laminate material.

Clause 4. The thrombectomy system of Clause 2 or 3, wherein the suction source is configured to displace the cover from the proximal portion of the opening.

Clause 5. The thrombectomy system of Clause 4, wherein the cover is configured to be displaced proximally by the suction source.

Clause 6. The thrombectomy system of any one of the preceding Clauses, wherein when the suction source is coupled with the tubular member, the suction source applies a greater negative pressure in the distal portion of the opening than in the proximal portion of the opening.

Clause 7. The thrombectomy system of any one of the preceding Clauses, wherein the proximal portion of the opening spans a first length of the tubular member and the distal portion of the opening spans a second length of the tubular member less than the first length.

Clause 8. The thrombectomy system of any one of the preceding Clauses, further comprising a core member extending through the lumen.

Clause 9. The thrombectomy system of any one of the preceding Clauses, wherein the distal end region of the tubular member comprises a plurality of circumferential struts, the circumferential struts defining the opening.

Clause 10. The thrombectomy system of any one of the preceding Clauses, wherein the circumferential struts have a greater circumferential width in a proximal section than in a distal section.

Clause 11. The thrombectomy system of any one of the preceding Clauses, wherein the circumferential struts comprise an upper portion and a lower portion, the upper portion offset longitudinally from the lower portion.

Clause 12. The thrombectomy system of any one of the preceding Clauses, wherein the circumferential struts are connected to one another by one or more intermediate struts.

Clause 13. The thrombectomy system of any one of the preceding Clauses, further comprising a plurality of protrusions extending from the circumferential struts.

Clause 14. The thrombectomy system of any one of the preceding Clauses, wherein the protrusions are configured to prevent a core member from exiting the lumen through the opening.

Clause 15. The thrombectomy system of any one of the preceding Clauses, wherein the protrusions are configured to engage the thrombus.

Clause 16. The thrombectomy system of any one of the preceding Clauses, wherein the tubular member further comprises a distal opening on a distal face of the tubular member.

Clause 17. A method comprising: disposing a medical device within a vessel at or adjacent a treatment site, the medical device comprising: a tubular member, the tubular member having a side opening in a distal region of the tubular member, wherein the side opening comprises a proximal portion and a distal portion; and applying a negative pressure to the tubular member, wherein the negative pressure is greater in the distal portion of the side opening than in the proximal portion of the side opening for a first time period, and wherein the negative pressure in the distal portion of the side opening is less than or equal to the negative pressure in the proximal portion of the side opening for a second time period after the first time period.

Clause 18. The method of any one of the preceding Clauses, wherein the medical device further comprises a cover overlying the proximal portion of the side opening.

Clause 19. The method of any one of the preceding Clauses, wherein, during the first time period, the cover is in a first position, and during the second time period, the cover is in a second position proximal to the first position.

Clause 20. The method of any one of the preceding Clauses, wherein during the second time period, the cover is at least partially displaced.

Clause 21. The method of any one of the preceding Clauses, wherein the negative pressure is supplied by a suction source.

Clause 22. The method of any one of the preceding Clauses, wherein the proximal portion of the side opening occupies a first length of the tubular member and the distal portion of the side opening occupies a second length of the tubular member less than the first length.

Clause 23. The method of any one of the preceding Clauses, wherein the medical device further comprises a core member extending through a lumen of the tubular member.

Clause 24. The method of any one of the preceding Clauses, wherein the distal region of the tubular member comprises a plurality of circumferential struts, the circumferential struts defining the side opening.

Clause 25. The method of any one of the preceding Clauses, wherein the circumferential struts comprise an upper portion and a lower portion, the upper portion offset longitudinally from the lower portion.

Clause 26. The method of any one of the preceding Clauses, wherein the circumferential struts are connected to one another by one or more intermediate struts.

Clause 27. The method of any one of the preceding Clauses, wherein the medical device further comprises a plurality of protrusions extending from the circumferential struts.

Clause 28. The method of any one of the preceding Clauses, wherein the protrusions are configured to prevent the core member from exiting the lumen of the tubular member through the side opening.

Clause 29. The method of any one of the preceding Clauses, wherein the protrusions are configured to engage a thrombus at the treatment site.

Clause 30. A thrombectomy device comprising: a tubular member having a sidewall defining a lumen extending therethrough from a proximal end region to a distal end region, the tubular member configured to be fluidically coupled to a suction source to supply negative pressure to the lumen; an opening in the sidewall in the distal end region, the opening including a first portion and a second portion; and a rupturable cover extending over only the first portion of the opening such that the second portion of the opening is uncovered.

Clause 31. The thrombectomy device of any one of the preceding Clauses, wherein the first portion is proximal to the second portion.

Clause 32. The thrombectomy device of any one of the preceding Clauses, wherein the first portion has a smaller width than the second portion.

Clause 33. The thrombectomy device of any one of the preceding Clauses, wherein the first portion has a longer axial length than the second portion.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 shows a perspective view of a thrombectomy system for retrieving material from a body lumen, in accordance with one or more embodiments of the present technology.

FIG. 2A illustrates an example distal end portion of the thrombectomy system of FIG. 1.

FIG. 2B illustrates another example distal end portion of the thrombectomy system of FIG. 1.

FIG. 2C illustrates an example distal end region of a thrombectomy device of the thrombectomy system shown in FIG. 2B, in accordance with embodiments of the present technology.

FIG. 3 shows an example distal end region of a thrombectomy device, in accordance with embodiments of the present technology.

FIG. 4 shows another example distal end region of a thrombectomy device, in accordance with embodiments of the present technology.

FIG. 5 shows a further example distal end region of a thrombectomy device, in accordance with embodiments of the present technology.

FIGS. 6A-6C illustrate an example method of deploying a thrombectomy device into a vessel, in accordance with embodiments of the present technology.

FIGS. 7A-7C illustrate another example method of deploying a thrombectomy device into a vessel, in accordance with embodiments of the present technology.

### DETAILED DESCRIPTION

The present technology relates to thrombectomy systems, devices, and methods for treating vascular obstructions, such as vessel occlusions, and associated devices and methods. In some embodiments, for example, a thrombectomy device includes a tubular member that can facilitate removal of a thrombus using aspiration to pull the thrombus into the tubular member lumen. In contrast to conventional aspiration catheters with a distal opening that is coaxial with the catheter lumen, in at least some embodiments of the present technology the tubular member can include an elongated opening in the sidewall instead of or in addition to a distal opening that is coaxial with the lumen. Additionally, the elongated sidewall opening can have a geometry such that the open space defined by the elongated sidewall opening is greater in the distal portion of the opening than in the proximal portion (e.g., the opening width can be greater in the distal portion than in the proximal portion). In operation, the tubular member can be disposed adjacent to a thrombus and suction can be applied to the tubular member lumen (e.g., via an extracorporeal pump or other suitable approach). The thrombus can then be adhered to the sidewall opening and/or pulled partially or completely into the lumen through the sidewall opening.

In some implementations, a temporary cover (e.g., a rupturable or separable polymer material) can be disposed over the sidewall opening along only the proximal portion, leaving the wider distal portion of the opening uncovered. In such configurations, when the tubular member is disposed adjacent to a thrombus and suction is applied, the thrombus may initially adhere to the uncovered distal portion of the sidewall opening, and at least a portion of the thrombus may begin to move through the distal portion of the sidewall opening into the lumen. In doing so, the fluid flow through the distal portion of the sidewall opening may decrease, thereby exerting increased pressure on the temporary cover over the proximal portion of the sidewall opening. The temporary cover can be configured to rupture, peel, separate, or otherwise become displaced, thereby uncovering the proximal portion of the opening. In some instances, this displacement can occur gradually in the distal-to-proximal direction, creating a zipper-like effect in which the proximal portion of the sidewall opening is gradually uncovered. As this proximal portion is uncovered, the thrombus can adhere to and/or be drawn into the lumen through the proximal portion of the sidewall opening.

The systems of the present technology can provide many advantages compared to conventional devices for treating vascular obstructions. The opening in the sidewall of the tubular member enables the system to engage multiple portions of the clotting material simultaneously or substantially simultaneously. In contrast, conventional systems typically include an aspiration catheter with only an opening on a distal face of the aspiration catheter. As a result, conventional systems have limited engagement with clotting material (e.g., engagement of only a proximal end portion of the clotting material). Conventional aspiration catheters are also susceptible to “corking,” in which the thrombus completely blocks the distal opening of the aspiration catheter, but the clot is unable to be pulled into the catheter lumen for removal. Moreover, such “corking” only grasps a small area of the thrombus (e.g., only a proximal-most portion), and accordingly attempts to retract the aspiration catheter with the corked thrombus intact often leads to clot fragmentation, risking downstream embolization. This problem is particularly acute in the small vessels of the neurovasculature, as the small catheters required for accessing these vessels necessarily have small lumens. Improved thrombus engagement, as provided with the present technology, reduces the risk of clot fragmentation, snaring of the thrombectomy system, further occlusion of vessels, arrest of flow rate, and more. Additionally, the tubular member, as further described herein, can have a slim profile with a low delivery force, reducing the risk of vessel perforation and/or damage. In some embodiments, the opening in the sidewall is configured such that suction is preferentially applied in a distal region of the clotting material. Further advantages will be made apparent with reference to embodiments of the present technology.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

### I. OVERVIEW OF EXAMPLE TREATMENT SYSTEMS AND DEVICES

The present technology provides systems, devices, and methods for removing clot material from a blood vessel lumen. Although many of the embodiments are described below with respect to devices, systems, and methods for treating a cerebral or intracranial embolism, other applications and other embodiments in addition to those described herein are within the scope of the technology. For example, the treatment systems and methods of the present technology may be used to remove emboli from body lumens other than blood vessels (e.g., the digestive tract, etc.) and/or may be used to remove emboli from blood vessels outside of the brain (e.g., pulmonary, abdominal, cervical, or thoracic blood vessels, or peripheral blood vessels including those within the legs or arms, etc.). In addition, the treatment systems and methods of the present technology may be used to remove luminal obstructions other than clot material (e.g., plaque, resected tissue, foreign material, etc.).

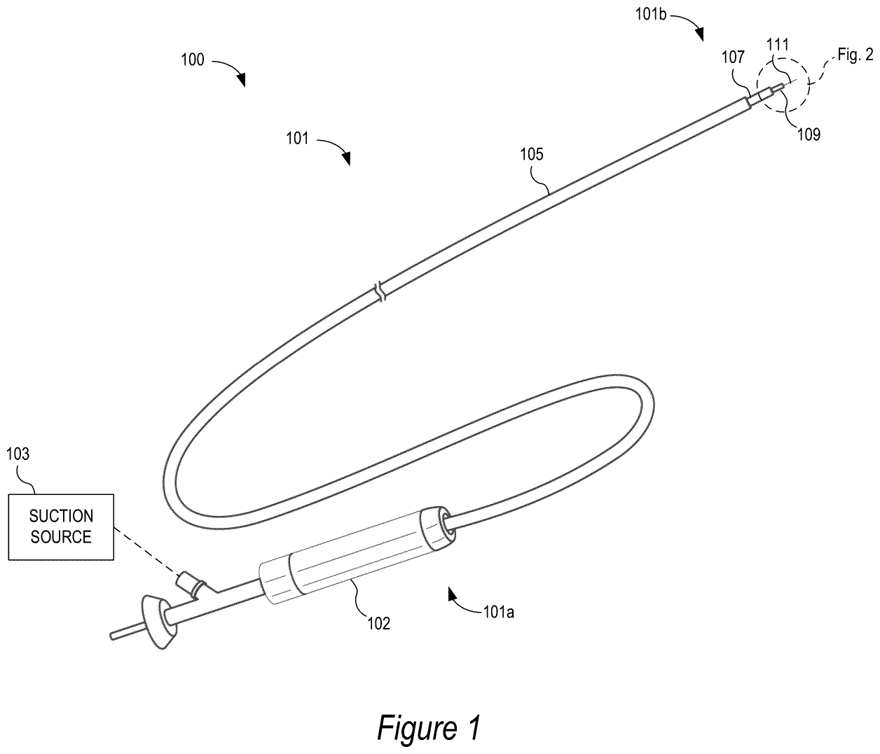

FIG. 1 illustrates a view of a thrombectomy system 100 according to one or more embodiments of the present technology. As shown in FIG. 1, the thrombectomy system 100 can include a medical device assembly 101 and a suction source 103. The medical device assembly 101 includes a proximal portion 101a configured to be coupled to the suction source 103 and a distal portion 101b configured to be intravascularly positioned within a blood vessel (such as an intracranial blood vessel) at a treatment site at or proximate a thrombus. The medical device assembly 101 includes a handle 102 at the proximal portion 101a. A plurality of elongated shafts or tubular members extend between the proximal portion 101a and the distal portion 101b. For example, in some embodiments, such as that shown in FIG. 1, the medical device assembly 101 includes a first or guide catheter 105 (e.g., a balloon-guide catheter), a distal access catheter 107 configured to be slidably disposed within a lumen of the guide catheter 105, a thrombectomy device 109 in the form of a tubular member (e.g., an aspiration catheter) configured to be slidably disposed within a lumen of the distal access catheter 107, and a guidewire or guidewire 111 configured to be slidably disposed within a lumen of the thrombectomy device 109. In some embodiments, the medical device assembly 101 does not include one or more of the guide catheter 105, distal access 107, thrombectomy device 109, or the guidewire 111.

In operation, one or more of the guide catheter 105, the distal access catheter 107, and thrombectomy device 109 can be used as an aspiration catheter to remove a clot or other material such as plaques or foreign bodies from vasculature of a patient. For example, a vacuum may be applied to proximal end of the thrombectomy device 109 (e.g., via suction source 103) to draw a clot or other blockage into an inner lumen of the thrombectomy device 109. In some embodiments, the vacuum causes the clot or other blockage to remain attached to the thrombectomy device 109 (e.g., on an outer surface of the thrombectomy device 109). Such aspiration may be used in various medical procedures, such as a medical procedure to treat an ischemic insult, which may occur due to occlusion of a blood vessel (arterial or venous) that deprives brain tissue, heart tissue or other tissues of oxygen-carrying blood.

With continued reference to FIG. 1, in some examples, the thrombectomy device 109 can be configured to access relatively distal locations in a patient including, for example, the middle cerebral artery (MCA), internal carotid artery (ICA), the Circle of Willis, and tissue sites more distal than the MCA, ICA, and the Circle of Willis. The MCA, as well as other vasculature in the brain or other relatively distal tissue sites (e.g., relative to the vascular access point), may be relatively difficult to reach with a tubular member, due at least in part to the tortuous pathway (e.g., comprising relatively sharp twists or turns) through the vasculature to reach these tissue sites. As such, the tubular member may be structurally configured to be relatively flexible, pushable, and relatively kink- and buckle-resistant, so that it may resist buckling when a pushing force is applied to a relatively proximal section of the tubular member to advance the tubular member distally through vasculature, and so that it may resist kinking when traversing around a tight turn in the vasculature. In some examples, the tubular member is configured to substantially conform to the curvature of the vasculature. In addition, in some examples, the tubular member has a column strength and flexibility that allow at least a distal portion of the tubular member to be navigated from a femoral artery, through the aorta of the patient, and into the intracranial vascular system of the patient, e.g., to reach a relatively distal treatment site.

Although primarily described as being used to reach relatively distal vasculature sites, the thrombectomy device 109 may also be configured to be used with other target tissue sites. For example, thrombectomy device 109 may be used to access tissue sites throughout the coronary and peripheral vasculature, the gastrointestinal tract, the urethra, ureters, fallopian tubes, veins and other body lumens.

According to some embodiments, the guide catheter 105 and the distal access catheter 107 can each be formed as additional tubular members extending along and about a central axis and terminating in respective distal ends. According to some embodiments, the distal access catheter 107 is generally constructed to track over the guidewire 111 in the cervical anatomy and into the cerebral vessels associated with the brain and may also be chosen according to several standard designs that are generally available. Accordingly, the distal access catheter 107 can have a length that is at least 125 cm long, and more particularly may be between about 125 cm and about 175 cm long.

The thrombectomy device 109 can be sized and configured to be slidably advanced over the guidewire 111. As noted above, the thrombectomy device 109 can be coupled at a proximal portion to a suction source 103 such as a pump or syringe in order to supply negative pressure to a treatment site. In various embodiments, the thrombectomy device 109 can have a length that is at least 125 cm long, and more particularly may be between about 125 cm and about 175 cm long. In some embodiments, the thrombectomy device 109 can be an aspiration catheter. The thrombectomy device 109 can have a lumen diameter of a between about 0.05″ and about 0.09″, for example about 0.061″, about 0.068″, or about 0.071″ lumen diameter. The tubular member 107 can have a maximum outer diameter of between about 0.06″ to about 0.1″, for example about 0.083″, or about 0.0855″. Other designs and dimensions are contemplated.

The guide catheter 105 can be sized and configured to slidably receive both the distal access catheter 107 and the thrombectomy device 109 therethrough. In some embodiments, the guide catheter 105 is a balloon-guide catheter having an inflatable balloon or other expandable member that can be used to anchor the guide catheter 105 with respect to a surrounding vessel. In operation the guide catheter 105 can first be advanced through a vessel and then a balloon can be expanded to anchor the guide catheter 105 in place and/or arrest blood flow from areas proximal of the balloon. Next, the distal access catheter 107 and the thrombectomy device 109 can be advanced together through the guide catheter 105 until they each extend distally beyond the distal end of the guide catheter 105. Suction can then be applied to aspirate the treatment site.

According to some embodiments, the bodies of the catheter 105, distal access catheter 107, and/or thrombectomy device 109 can be made from various thermoplastics, e.g., polytetrafluoroethylene (PTFE or TEFLON®), fluorinated ethylene propylene (FEP), high-density polyethylene (HDPE), polyether ether ketone (PEEK), etc., which can optionally be lined on the inner surface of the catheters and/or tubular member or an adjacent surface with a hydrophilic material such as polyvinylpyrrolidone (PVP) or some other plastic coating. Additionally, either surface can be coated with various combinations of different materials, depending upon the desired results. As described in more detail below, some or all of the thrombectomy device 109 can be formed of a metallic material, such as Nitinol, stainless steel, or other suitable material. In some examples, the thrombectomy device 109 can include a laser-cut hypotube having a pattern of cut voids (e.g. spiral cut, separated slot cuts, or other suitable pattern) formed in its sidewall along at least a portion of its length. In at least some embodiments, the thrombectomy device 109 can have a laser cut pattern to achieve the desired mechanical characteristics (e.g., column strength, flexibility, kink-resistance, etc.).

In various embodiments, the guidewire 111 can be a solid pushwire or guidewire. Additionally or alternatively, the guidewire 111 can instead include a hollow wire, hypotube, braid, coil, or other suitable member(s), or a combination of wire(s), tube(s), braid(s), coil(s), etc. In some embodiments, the guidewire 111 can be made of stainless steel (e.g., 304 SS), Nitinol, and/or other alloy.

### II. SELECT EXAMPLES OF THROMBECTOMY DEVICES HAVING DISTAL SIDE OPENINGS

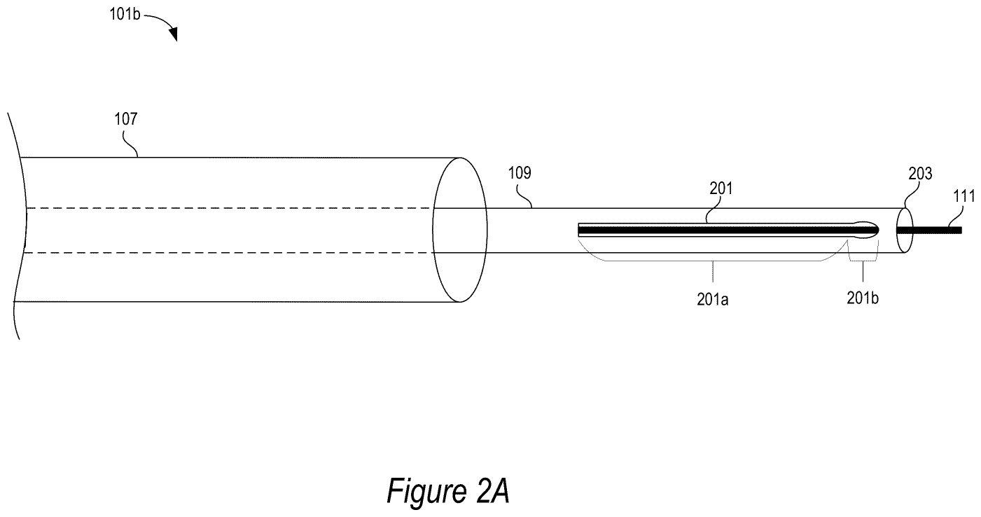

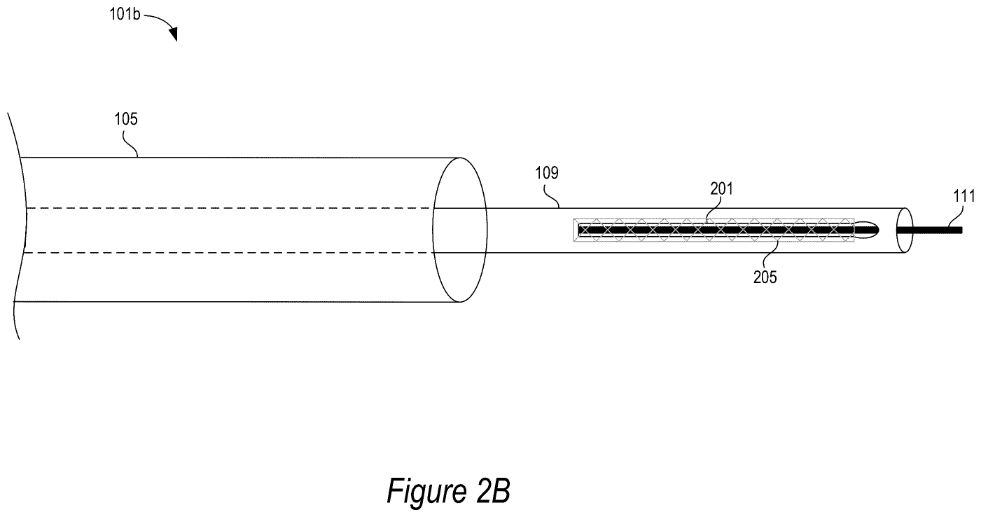

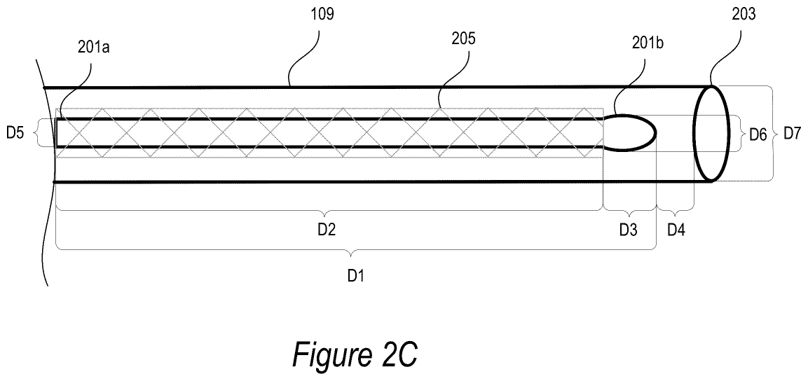

FIGS. 2A-2C illustrate example enlarged detail views of the distal portion 101b of the thrombectomy system 100 of FIG. 1. Referring first to FIG. 2A, the thrombectomy system 100 is configured to be deployed at an intravascular treatment site (e.g., at or adjacent to a thrombus). A guidewire 111 slidably extends through a lumen of the thrombectomy device 109, which in turn slidably extends through a lumen of the surrounding catheter 107. As noted elsewhere herein, the thrombectomy system 100 can include a suction source 103, such that when suction is applied, the thrombectomy device 109 is configured to engage clotting material.

In some embodiments, the thrombectomy device 109 is a tubular member having an opening 201 in a sidewall of the tube. The opening 201 can be configured to fluidically couple the lumen of the thrombectomy device 109 with the surrounding environment. The opening 201 can have any variety of geometries. For example, the opening 201 can be substantially oblong. Alternatively, or in addition, the opening 201 can include straight, arcuate, curved, semi-circular, or semi-elliptical shapes. The opening 201 can also include complex shapes, such as zig-zag, undulating, serpentine, sinusoidal, or a combination thereof.

As shown in FIG. 2A, the opening 201 can include a proximal portion 201a and a distal portion 201b. In some embodiments, the proximal portion 201a spans a greater length of the thrombectomy device 109 than the distal portion 201b. Further, the proximal portion 201a can span a smaller radial or circumferential width of the thrombectomy device 109 than the distal portion 201b. In some embodiments, a smaller length and a greater radial or circumferential width in the proximal portion 201a advantageously allows the thrombectomy device 109 to facilitate a greater flow rate in the distal portion 201b than in the proximal portion 201a when the thrombectomy device 109 is coupled to a suction source. While the opening 201 is depicted as having portions of two different shapes, the opening 201 can include any number of different shapes, e.g., at least one, two, three, four, five, six, seven, eight, nine, or ten shapes. Alternatively, or in combination, the opening 201 can be tapered from the proximal portion 201a to the distal portion 201b.

In some embodiments, the opening 201 is a continuous opening. For example, in some embodiments, the proximal portion 201a and the distal portion 201b are connected (e.g., span an unoccupied surface area of the thrombectomy device 109). Alternatively, or in combination, the first opening 201 can be a first opening 201 of a plurality of openings in the sidewall of the thrombectomy device 109. For example, the first opening 201 can occupy a first surface area and a second opening (not depicted) of the plurality of openings can occupy a second surface area different from the first surface area. In such cases, the first opening 201 and the second opening are separated by at least a portion of the thrombectomy device 109.

The thrombectomy device 109 can further include a distal opening 203 (e.g., a distal-facing mouth at the distal end of the thrombectomy device 109). In some embodiments, the distal opening 203 is configured to permit passage of the guidewire 111 therethrough, allowing the thrombectomy device 109 to be slidably advanced over the guidewire 111. Additionally, or alternatively, the distal opening 203 can be configured to aspirate clotting material. For example, when the thrombectomy device 109 is coupled to the suction source 103, the thrombectomy device 109 can engage clotting material via the distal opening 203. In some embodiments, engaging the clotting material via the distal opening 203 includes aspirating remnants of clotting material not engaged via the opening 201. A distal end of the sidewall opening 201 and the distal opening 203 can be separated from one another along the longitudinal axis of the thrombectomy device 109 by at least 1 mm, 2 mm, 3 mm, 4 mm, 5 mm, 10 mm, 20 mm, 50 mm, 100 mm, 1 cm, etc.

As depicted in FIGS. 2B and 2C, in some embodiments the opening in the sidewall of the thrombectomy device 109 is at least partially covered. Referring now to FIG. 2B, opening 201 is at least partially covered by a cover 205. Among examples, the proximal portion 201a of the sidewall opening 201 may be covered via the cover 205, while the distal portion 201b of the sidewall opening may be uncovered. The cover 205 can be configured to temporarily prevent passage of material from the environment into the thrombectomy device 109. In some embodiments, the cover 205 covers proximal portion 201a of the opening 201. For example, the cover 205 can cover at least 10%, 20%, 30%, 40%, 50%, 60%, 70%, 80%, 90%, or 100% of the proximal portion 201a. Alternatively, or in combination, the cover 205 can cover at least some of distal portion 201b of the opening 201. For example, the cover 205 can cover at least 10%, 20%, 30%, 40%, 50%, 60%, 70%, 80%, 90%, or 100% of the distal portion 201b.

The cover 205 can be attached to an external surface of the thrombectomy device 109. For example, the cover 205 can be connected to the thrombectomy device 109 by one or more of bonding, adhesives, welding, etc. Alternatively, or in combination, the cover 205 can be attached to an internal surface of the thrombectomy device 109.

The cover 205 can generally be configured to detach and/or release itself from the tubular member sidewall. In some embodiments, the cover 205 ruptures (e.g., breaks apart), tears, dissolves, or disintegrates, or otherwise ceases to cover the sidewall opening 201. The cover 205 can rupture in response to an engagement of the thrombectomy device 109 with clotting material. In some embodiments, the cover 205 ruptures in response to negative pressure applied by a suction source (e.g., the suction source 103), such that the cover 205 is at least partially displaced inwardly into the thrombectomy device 109. In some embodiments, the cover 205 ruptures along a midline of the cover 205. For example, the cover 205 may separate symmetrically about the longitudinal axis.

In some embodiments, the cover has a varying thickness. For example, the cover 205 can have a greater thickness in a proximal portion of the cover 205 than in a distal portion of the cover 205. In some embodiments, the cover 205 has a gradient thickness, e.g., a decreasing thickness from the proximal portion of the cover 205 to the distal portion of the cover 205. Varying the thickness of the cover 205 may advantageously cause the cover to rupture in a predetermined fashion. For example, when the cover 205 has a decreasing thickness from the proximal portion of the cover 205 to the distal portion of the cover 205, the cover 205 may rupture in the distal portion before the proximal portion. However, the cover 205 may be modified and/or configured in other ways that affect the manner in which the cover 205 ruptures. For example, the cover 205 may still preferentially rupture from the distal portion to the proximal portion while having a uniform thickness.

The cover can include any variety of materials. In some embodiments, the cover 205 includes one or more substantially impermeable material(s). For example, the cover 205 can include a laminate material. In some embodiments, the cover 205 includes PFTE, Pebax, polymers, etc. In some embodiments, the cover 205 includes a heat shrink material. Manufacture of the cover 205 may involve any number of lamination processes. The cover 205 can further be configured to be hydrophilic, e.g., having a phosphorocholine compound. Additionally, or alternatively, some or all of the cover 205 can include materials that enhance blood compatibility and/or reduces thrombogenic surface activity.

While the cover 205 is depicted as a continuous cover, in some embodiments, the cover 205 can include multiple segments. For example, the cover 205 can have a proximal segment, an intermediate segment, and a distal segment. In some embodiments, the proximal segment, the intermediate segment, and the distal segment are separated. For example, the proximal segment and the intermediate segment can be separated by at least 1 mm, 2 mm, 3 mm, 5 mm, 10 mm, 20 mm, 50 mm, 100 mm, 500 mm, etc. In some embodiments, any one of the proximal segment, the intermediate segment, and the distal segment can omitted. When the cover 205 comprises multiple segments, the cover 205 can selectively detach from the thrombectomy device 109. For example, in some embodiments, the distal segment of the cover 205 detaches from the thrombectomy device 109 before the proximal segment of the cover 205 detaches from the thrombectomy device 109.

FIG. 2C shows an enlarged detail view of the distal end region of the thrombectomy device 109, in which relative dimensions of the sidewall opening 201 and the cover 205 are shown. As illustrated, the sidewall opening 201 can span a first length D1. In some embodiments, the first length D1 is no more than 5 mm, 6 mm, 7 mm, 8 mm, 9 mm, 10 mm, 15 mm, 20 mm, 30 mm, 40 mm, or 50 mm. In some embodiments, the first length D1 is at least about 5 mm, 6 mm, 7 mm, 8 mm, 9 mm, 10 mm, 15 mm, 20 mm, 30 mm, 40 mm, or 50 mm. D1 can be the combined length of length D2, corresponding to the proximal portion 201a, and length D3, corresponding to the distal portion 201b.

Cover 205 can span a substantial portion of proximal portion 201a and/or distal portion 201b of the opening 201. For example, the cover 205 can span a length that is at least 10%, 20%, 30%, 40%, 50%, 60%, 70%, 80%, 90%, or 100% of D2 and/or no more than 10%, 20%, 30%, 40%, 50%, 60%, 70%, 80%, 90% of D3.

In some embodiments, the distal portion 201b of the opening 201 is separated by the distal cover 205 by a length D4. Length D4 can be smaller than lengths D1, D2, and/or D3. In some embodiments, length D4 is at least about 1 mm, 2 mm, 3 mm, 4 mm, 5 mm, 10 mm, 20 mm, 50 mm, 100 mm, 1 cm, etc. In some embodiments, length D4 is no more than about 1 mm, 2 mm, 3 mm, 4 mm, 5 mm, 10 mm, 20 mm, 50 mm, 100 mm, 1 cm, etc.

In some embodiments, proximal portion 201a of the opening 201 has a smaller diameter D5 than the diameter D6 of the distal portion 201b of the opening 201. For example, D5 can be no more than 10%, 20%, 30%, 40%, 50%, 60%, 70%, 80%, or 90% of D6. In some embodiments, D5 and D6 are the same, e.g., having equal diameters. Alternatively, D5 can be greater than D6, e.g., when the proximal portion 201a spans a greater radial width than the distal portion 201b.

Diameters D5 and D6 can be smaller than D7, the diameter of thrombectomy device 109. In some embodiments, the greater one of D5 and D6 is no more than 10%, 20%, 30%, 40%, 50%, 60%, 70%, 80%, or 90% of D7.

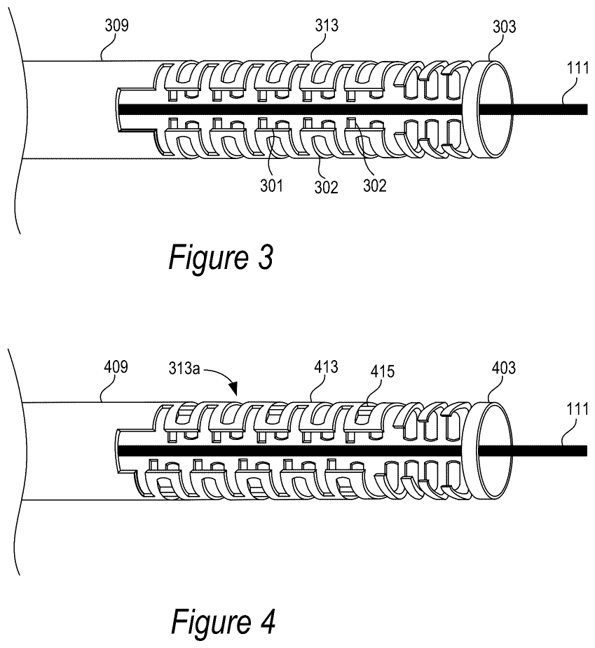

In some examples, the body of the tubular member of the thrombectomy device 109 can have cuts, recesses, grooves, struts, or other features that provide enhanced flexibility for improved navigation in the tortuous anatomy of the neurovasculature. FIG. 3 shows an example distal end region of a thrombectomy device including such struts, in accordance with embodiments of the present technology. As depicted, the distal end region of thrombectomy device 309 can include a plurality of circumferential struts 313. In some embodiments, the circumferential struts 313 have the same diameter as thrombectomy device 109. The circumferential struts 313 can span a different (e.g., smaller) arc length than the rest of the thrombectomy device 309. For example, the circumferential struts 313 can span an arc length that is no more than 10%, 20%, 30%, 40%, 50%, 60%, 70%, 80%, or 90% of the arc length (e.g., total circumference) of the thrombectomy device 109. In some embodiments, each of the circumferential struts 313 span the same arc length with a common central angle. However, in some embodiments, the arc length and/or central angle may vary across circumferential struts 313.

The circumferential struts 313 can be equally spaced apart along the longitudinal axis. For example, in some embodiments, the distance between neighboring circumferential struts 313 is no more than 2.5 mm, 2.25 mm, 2 mm, 1.75 mm, 1.5 mm, 1.25 mm, 1 mm, 0.75 mm, or 0.5 mm. Alternatively, the circumferential struts 313 can include a variety of spacings therebetween. For example, in some embodiments, intermediate spacings between the circumferential struts 313 increases distally such that the circumferential struts 313 are closer together in a proximal portion than in a distal portion. The circumferential struts 313 can include any number of circumferential struts. For example, the circumferential struts 313 can comprise at least one, two, three, four, five, six, seven, eight, nine, ten, 20, or more circumferential struts.

The circumferential struts can form a sidewall opening 301. For example, portions of the circumferential struts 313 that depart from the rest of the thrombectomy device 109 can form an opening 301 including a plurality of recesses and/or channels. The opening 301 can fluidically couple the lumen of the thrombectomy device 109 with the surrounding environment. In some embodiments, the opening 301 is configured to receive clotting material from the surrounding environment. The opening 301 can be generally similar to the opening 201 of FIGS. 2A-2C and provide similar advantages and/or functionalities.

In some embodiments, the circumferential struts are integral to the tubular member. For example, the circumferential struts 313 can be formed via the removal of material from the thrombectomy device 309. Alternatively, the circumferential struts 313 and the rest of the thrombectomy device 309 can be discrete components that are attached to each other, e.g., via welding, adhesives, fasteners, or other suitable techniques.

The circumferential struts 313 can have different material properties from the rest of the thrombectomy device 309. For example, the circumferential struts 313 can include one or more additional materials that affect a stiffness and/or elastic modulus of the circumferential struts 313. For instance, the circumferential struts 313 can include a lower stiffness than the rest of the thrombectomy device 309. In such cases, the circumferential struts 313 may more easily fold and/or collapse than the rest of the thrombectomy device 309, e.g., when one or more forces are imparted on the thrombectomy device 309.

In some embodiments, intermediate spacings between the circumferential struts 313 form a plurality of secondary openings 302. The circumferential struts 313 can be configured to compress and expand such that the secondary openings 302 increase or decrease in volume in response to movement of the thrombectomy device 109.

In some embodiments, the circumferential struts comprise an upper portion and a lower portion. Turning now to FIG. 4, an example distal end region of a thrombectomy device, in accordance with embodiments of the present technology, is shown. The distal end portion of tubular member 409 can include a plurality of circumferential struts 413 that are generally similar to the circumferential struts 313 of FIG. 3. In some embodiments, upper portion 413a and lower portion 413b are offset from one another. For example, the upper portion 413a can include circumferential struts that are distal to corresponding circumferential struts of the lower portion 413b by at least 1 mm, 2 mm, 3 mm, 4 mm, 5 mm, 10 mm, 20 mm, 50 mm, 100 mm, 1 cm, etc. In some embodiments, offsetting the lower portion 413b from the upper portion 413a, or vice-versa, advantageously provides greater flexibility and maneuverability of the thrombectomy device 109.

In some embodiments, at least some of the circumferential struts 413 are attached to one another by one or more intermediate struts 415. The intermediate struts 415 can have straight, arcuate, curved, semi-circular, or semi-elliptical shapes. The intermediate struts 415 may connect a more proximally located circumferential strut to a more distally located circumferential strut. In some embodiments, connecting two or more circumferential struts 413 with intermediate struts 415 advantageously provides greater support and may allow for easier deployment of the thrombectomy device 109 in a vessel of a patient.

The intermediate struts 415 can be a separate component from the circumferential struts 413 or can be an integral component of the circumferential struts 413. In some embodiments, the intermediate struts 415 have a smaller thickness than the circumferential struts 413. Optionally, the intermediate struts 415 may include one or more different materials from the circumferential struts 413 that affect the stiffness and/or elastic modulus of the intermediate struts 415. For example, the intermediate struts 415 may include materials that result in the intermediate struts 415 having a lower stiffness than the circumferential struts 413.

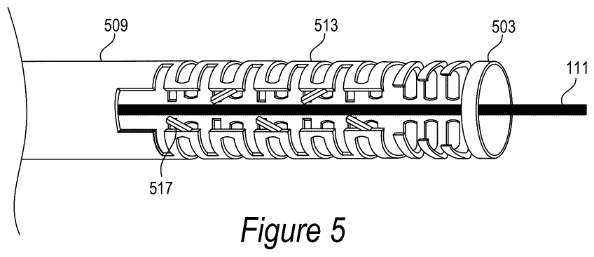

In some embodiments, one or more protrusions extend from the circumferential struts. Turning now to FIG. 5, one or more protrusions 517 extend from circumferential struts 513. The one or more protrusions 517 can include one or more of tines, barbs, fixation members, hooks, rods, elongate members, or any other suitable engagement bodies. The one or more protrusions 517 can extend from the circumferential struts 513 at an angle from about 0 to 10 degrees, 10 to 20 degrees, 30 to 40 degrees, 40 to 50 degrees, 50 to 60 degrees, 70 to 80 degrees, or 80 to 90 degrees.

The one or more protrusions 517 may serve multiple purposes. For example, the one or more protrusions 517 can be configured to prevent guidewire 511 from leaving a lumen of the thrombectomy device 509. For instance, the protrusions 517 can form retention members configured to retain the guidewire 511 within the lumen of the thrombectomy device 509. In some embodiments, the one or more protrusions 517 are configured to engage the clotting material by one or more of displacing, penetrating, catching, grappling, or entrapping the clotting material. As will be discussed further below, the protrusions 517 can be configured to engage the clotting material while the thrombectomy device 509 is removed from a bodily lumen.

In some embodiments, the one or more protrusions 517 are coupled to more than one circumferential strut. For example, a protrusion 517 can extend from a circumferential strut to a neighboring circumferential strut. In such cases, the protrusion 517 can act as a guarding structure to prevent the guidewire 111 from leaving the lumen of the thrombectomy device 509.

### III. EXAMPLE THROMBECTOMY METHODS

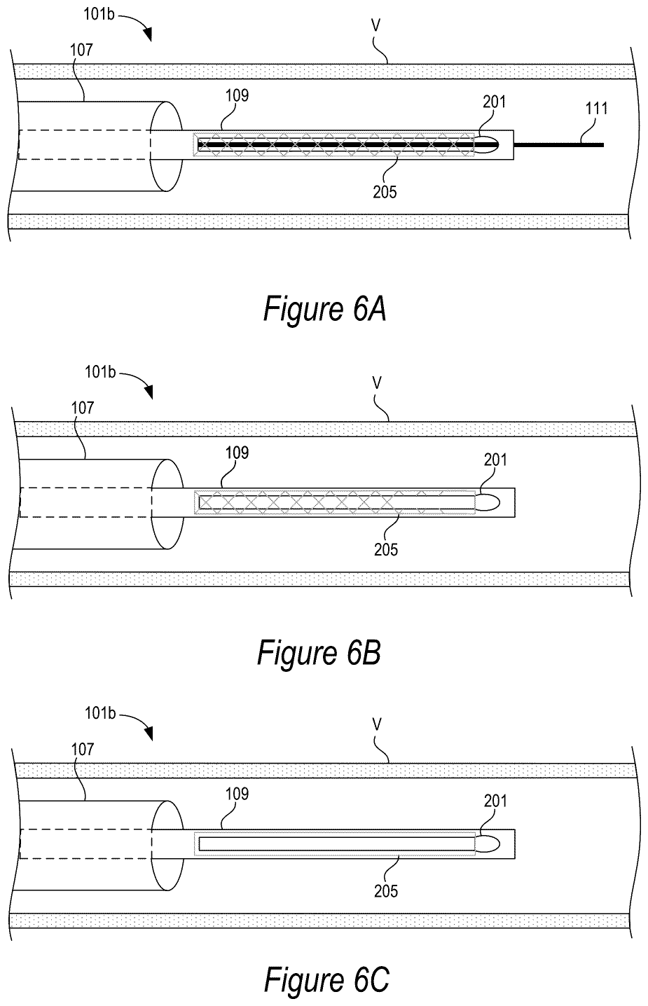

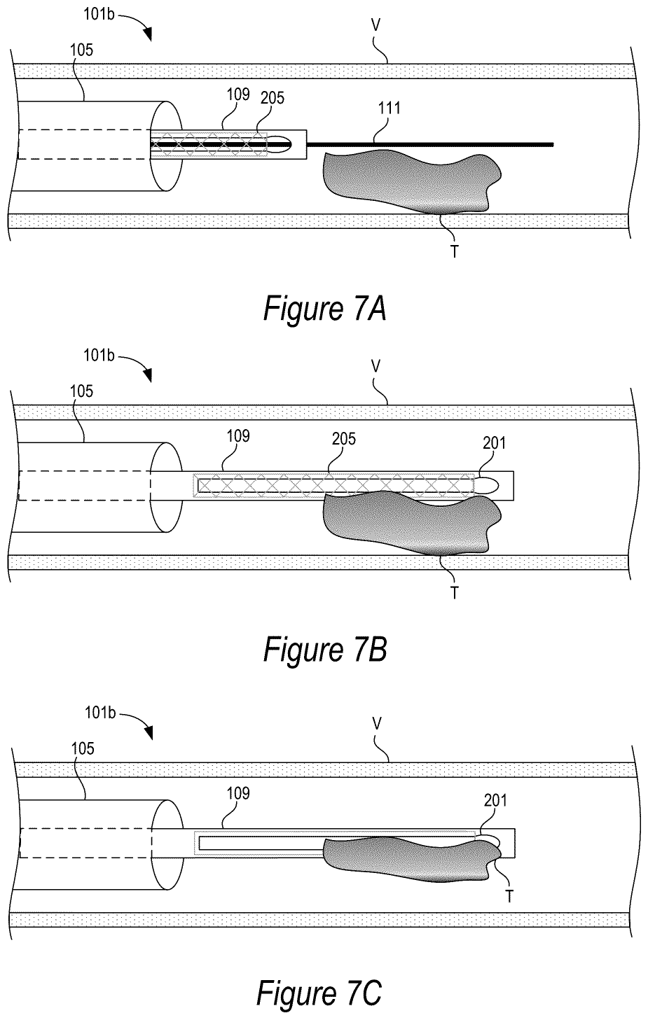

The thrombectomy devices and systems provided herein can be used in treating a variety of vessel occlusions. Although the methods are described herein primarily with reference to a thrombectomy device with Figure-specific reference numbers for clarity, it should be understood that the methods described herein may additionally or alternatively be performed with any suitable variation of thrombectomy devices in accordance with the present technology (e.g., thrombectomy system 100), such as those described above with respect to FIGS. 1A-5. Some methods will now be illustrated with respect to FIGS. 6A-7C.

FIGS. 6A-6C illustrate an example method of deploying a thrombectomy system into a vessel, in accordance with embodiments of the present technology. The thrombectomy system 100 can include thrombectomy device 109 having a sidewall opening 201 and cover 205.

Referring now to FIG. 6A, a guidewire 111 is distally advanced in a blood vessel V, followed by the distal advancement of a catheter 107 and/or thrombectomy device 109. In some embodiments, the thrombectomy device 109 is distally advanced within the guide catheter 105. Alternatively, or in combination, the catheter 107 may be retracted at a treatment site, leaving the thrombectomy device 109 in place and at least partially outside a lumen of the guide catheter 105. The cover 205 may be coupled to the thrombectomy device 109 for the entirety of the distal advancement of the thrombectomy device 109 to the treatment site. After the thrombectomy device 109 is placed at the treatment site, guidewire 111 may be removed, e.g., proximally withdrawn. Optionally, the catheter 107 can similarly be withdrawn from the blood vessel V.

In some embodiments, the thrombectomy system 100 includes a suction source (not depicted). The suction source can be coupled to the thrombectomy device 109 and is configured to apply negative pressure within a lumen of the thrombectomy device 109. When the suction source is coupled to the thrombectomy device 109, clot material can be pulled against the uncovered portion of the sidewall opening 201. In some instances, this engagement can result in a “corking” effect, in which fluid flow through the sidewall opening 201 is temporarily occluded. As suction continues to be applied, the suction source can initiate a rupturing of the cover 205. For example, when the suction source applies suction to the thrombectomy device 109, the cover 205 can begin to rupture from the distal end to the proximal end, as previously discussed herein.

FIG. 6B shows the cover 205 in a partially ruptured state. In some embodiments, wherein the cover 205 is a mesh, rupturing the cover 205 includes tearing and/or ripping off the mesh. In some embodiments, wherein the cover 205 is a single continuous layer, the cover 205 is separated along a midline of the cover 205. In response to one or more forces imparted by the suction source or other external energy source, the cover 205 can be displaced inwardly to a lumen of the thrombectomy device 109. As the cover 205 is ruptured, fluid flows (due to the applied suction) through the uncovered portions of the sidewall opening 201, causing the clot material to be adhered to those uncovered portions of the opening. As such, the engagement area between the clot and the sidewall opening 201 increases as the cover 205 is ruptured.

In some embodiments, the cover 205 is aspirated proximally such that the cover 205 is displaced by the suction source and leaves the opening 201 unobstructed. For example, when the cover 205 ruptures and is no longer attached to the thrombectomy device 109, the opening 201 can fluidically couple the lumen of the thrombectomy device 109 with the surrounding environment, as depicted in FIG. 6C.

FIGS. 7A-7C illustrate another example method of deploying a thrombectomy device into a vessel, in accordance with embodiments of the present technology. The thrombectomy system 100 can include thrombectomy device 109 having an opening 201 and a cover 205.

Referring now to FIG. 7A, guidewire 111 is advanced distally in a blood vessel V, followed by the distal advancement of guide catheter 105 and/or thrombectomy device 109. In some embodiments, the thrombectomy device 109 is distally advanced within guide catheter 105. Alternatively, or in combination, the guide catheter 105 may be retracted at a treatment site, leaving the thrombectomy device 109 in place and at least partially outside a lumen of the guide catheter 105. The cover 205 may be coupled to the thrombectomy device 109 for the entirety of the distal advancement of the thrombectomy device 109 to the treatment site. After the thrombectomy device 109 is placed at the treatment site, guidewire 111 may be removed, e.g., proximally withdrawn. Optionally, the guide catheter 105 can similarly be withdrawn from the blood vessel V.

In some embodiments, the thrombectomy device 109 is rotated such that the opening 201 faces the thrombus T. For example, a handle (not shown) may be coupled to the thrombectomy device 109 at a proximal end region of the thrombectomy device 109. An operator, e.g., a physician, can rotate the handle to assume a favorable position for aspirating clotting material from the blood vessel V. The thrombectomy device 109 can be coupled to a suction source (not shown). The suction source can be configured to cause negative pressure in the lumen of the thrombectomy device 109.

After the thrombectomy device 109 is appropriately placed at or adjacent to the thrombus T, as depicted in FIG. 7B, the cover can begin to rupture. For example, in some embodiments, when the suction source is coupled to the thrombectomy device 109, the cover 205 begins to rupture from the distal end to the proximal end. Negative pressure applied by the suction source can cause the distal end of the cover 205 to break apart, increasing the size of the opening 201. In some embodiments, the cover 205 is displaced proximally. In some embodiments, the cover 205 is displaced into the lumen of the thrombectomy device 109.

After the cover 205 is removed from the thrombectomy device 109, the suction source can continue to provide negative pressure to the lumen of the thrombectomy device 109. In some embodiments, the suction source is configured to cause the thrombectomy device 109 to engage the thrombus T at the opening 201. For example, negative pressure provided by the suction source can cause the thrombus T to be at least partially drawn inward to the lumen of the thrombectomy device 109. In some embodiments, the opening 201 is configured to engage the thrombus T without the thrombus T entering the lumen of the thrombectomy device 109. For example, the opening 201 can include a plurality of protrusions (e.g., such as the protrusions 517 of FIG. 5).

After the thrombus T engages the thrombectomy device 109 at the opening 201, the thrombectomy device 109 can be retracted. In some embodiments, the thrombectomy device 109 is completely removed from the blood vessel V. Alternatively, or in combination, the thrombectomy device 109 can break apart the thrombus T. For example, when the thrombectomy device 109 includes one or more protrusions, the protrusions can fragment the thrombus T into smaller pieces suitable for aspiration. Fragmentation may be desirable when the thrombus T is too large to be aspirated.

Further still, aspiration can occur at the opening 201 or a distal opening on the distal face of the thrombectomy device 201. In some embodiments, aspirating from both the opening 201 and the distal opening advantageously prevents clotting material from moving further downstream after it engages the thrombectomy device 201.

The methods of the present technology can be performed under fluoroscopy such that at least some portions of the thrombectomy device can be visualized by a physician to ensure proper placement of the thrombectomy device. For example, the thrombectomy device can include one or more radiopaque portions or markers (e.g., on a distal end of the tubular member). The one or more radiopaque portions can be visualized using fluoroscopy and/or other suitable imaging techniques to assist in positioning the thrombectomy device.

### IV. CONCLUSION

Although many of the embodiments are described above with respect to systems, devices, and methods for treating vessel occlusions in the brain, the technology is applicable to other applications and/or other approaches, such as vessel occlusions elsewhere in the body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-7C.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent embodiments in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombectomy system comprising:
a thrombectomy device including a tubular member having a proximal end region configured to be disposed extracorporeally and a distal end region configured to be disposed at an intravascular treatment site at or adjacent to a thrombus, the tubular member comprising:
a sidewall defining a lumen extending therethrough from the proximal end region to the distal end region; and
an opening in the sidewall in the distal end region, the opening including a proximal portion spanning a first width and a distal portion spanning a second width greater than the first width; and
a suction source configured to be fluidically coupled to the lumen.

2. The thrombectomy system of claim 1, further comprising a cover at least partially overlying the proximal portion of the opening.

3. The thrombectomy system of claim 2, wherein the suction source is configured to displace the cover from the proximal portion of the opening.

4. The thrombectomy system of claim 1, wherein when the suction source is coupled with the tubular member, the suction source applies a greater negative pressure in the distal portion of the opening than in the proximal portion of the opening.

5. The thrombectomy system of claim 1, wherein the proximal portion of the opening spans a first length of the tubular member and the distal portion of the opening spans a second length of the tubular member less than the first length.

6. The thrombectomy system of claim 1, wherein the distal end region of the tubular member comprises a plurality of circumferential struts, the circumferential struts defining the opening.

7. The thrombectomy system of claim 6, wherein the circumferential struts have a greater circumferential width in a proximal section than in a distal section.

8. The thrombectomy system of claim 6, wherein the circumferential struts comprise an upper portion and a lower portion, the upper portion offset longitudinally from the lower portion.

9. The thrombectomy system of claim 6, further comprising a plurality of protrusions extending from the circumferential struts.

10. The thrombectomy system of claim 1, wherein the tubular member further comprises a distal opening on a distal face of the tubular member.

11. A method comprising:
disposing a medical device within a vessel at or adjacent a treatment site, the medical device comprising:
a tubular member, the tubular member having a side opening in a distal region of the tubular member, wherein the side opening comprises a proximal portion and a distal portion; and
applying a negative pressure to the tubular member, wherein the negative pressure is greater in the distal portion of the side opening than in the proximal portion of the side opening for a first time period, and wherein the negative pressure in the distal portion of the side opening is less than or equal to the negative pressure in the proximal portion of the side opening for a second time period after the first time period.

12. The method of claim 11, wherein the medical device further comprises a cover overlying the proximal portion of the side opening.

13. The method of claim 11, wherein, during the first time period, the cover is in a first position, and during the second time period, the cover is in a second position proximal to the first position.

14. The method of claim 11, wherein during the second time period, the cover is at least partially displaced.

15. The method of claim 11, wherein the proximal portion of the side opening occupies a first length of the tubular member and the distal portion of the side opening occupies a second length of the tubular member less than the first length.

16. The method of claim 11, wherein the distal region of the tubular member comprises a plurality of circumferential struts, the circumferential struts defining the side opening.

17. A thrombectomy device comprising:
a tubular member having a sidewall defining a lumen extending therethrough from a proximal end region to a distal end region, the tubular member configured to be fluidically coupled to a suction source to supply negative pressure to the lumen;
an opening in the sidewall in the distal end region, the opening including a first portion and a second portion; and
a rupturable cover extending over only the first portion of the opening such that the second portion of the opening is uncovered.

18. The thrombectomy device of claim 17, wherein the first portion is proximal to the second portion.

19. The thrombectomy device of claim 17, wherein the first portion has a smaller width than the second portion.

20. The thrombectomy device of claim 17, wherein the first portion has a longer axial length than the second portion.
